(19) United States
(12) Patent Application Publication
(10) Pub. No.: US 2012/0029066 A1
(43) Pub. Date: Feb. 2, 2012

(54) USE OF FLAVONES FOR TREATING PSYCHIATRIC DISORDERS WITH SENSORIMOTOR GATING DEFICITS

(75) Inventors: Lih-Chu CHIOU, Taipei-City (TW); Wei-Jan HUANG, Taipei-City (TW)

(73) Assignees: Taipei Medical University, Taipei-City (TW); National Taiwan University, Taipei-City (TW)

(21) Appl. No.: 13/195,001

(22) Filed: Aug. 1, 2011

Related U.S. Application Data
(60) Provisional application No. 61/369,089, filed on Jul. 30, 2010.

(51) Int. Cl. A61K 31/352; A61P 25/18; A61P 25/00; C07D 311/30

(52) U.S. Cl. 514/456; 549/403

(57) ABSTRACT

Novel Use of flavones having 2-phenylchromen-4-one skeleton is disclosed herein. The flavones are useful as lead compounds for manufacturing a medicament or a pharmaceutical composition for treating a patient diagnosed with a psychiatric disorder with sensorimotor gating deficits.

### CROSS-REFERENCE TO RELATED APPLICATION

This application claims priority to U.S. Provisional Application No. 61/369,089, filed Jul. 30, 2010, the entirety of which is incorporated herein by reference.

### BACKGROUND OF THE INVENTION

1. Field of the Invention

The present disclosure relates to novel use of flavones, particularly, flavones having 2-phenylchromen-4-one skeleton, for the treatment of psychiatric disorders, particularly neuropsychiatric disorders with sensorimotor gating deficits.

2. Description of Related Art

Prepulse inhibition (PPI) is an operational measure of sensorimotor gating and is impaired in a family of psychiatric disorders, particularly, neuropsychiatric disorders characterized by abnormalities of inhibitory gating function.

PPI is a neurophysiological phenomenon in which a weaker prestimulus (i.e., prepulse) inhibits the reaction of an organism to a subsequent strong startling stimulus, which could be a sound, an airpuff or a light. PPI is a fast neuroadaptative system to protect living organisms from startle stimulation. It is believed to be a processing protection in a living organism that PPI serves as a sensorimotor gating, a pre-conscious regulator of attention, to reduce the startle response which is harmful to the information professing. Deficits in PPI manifest in the inability to filter out the unnecessary information, and have been linked to abnormalities of sensorimotor gating. Such deficits are noted in patients suffering from illnesses such as schizophrenia, attention deficient hyperactivity disorder (ADHD), obsessive compulsive disorder (OCD) or Tourette's syndrome (TS), and leading to the use of PPI measurement as a preclinical model of such illnesses. In mice, PPI decreased in a manner that is homologous to that seen in subjects following administration of certain psycotomimetic drugs (e.g., methamphetamine (MeAmph) or ketamine (KET)).

In this study, inventors unexpectedly identified novel use of several flavones, each possesses a 2-phenylchromen-4-one skeleton and is effective in re-instating disruption of PPI caused by certain psycotomimetic drugs such as methamphetamine or N-methyl D-aspartate (NMDA) receptor channel blockers (e.g., ketamine or MK-801) in mice, hence these flavones are potential lead compounds for the development of medicaments useful for the treatment of neuropsychiatric disorders with sensorimotor gating deficits.

### SUMMARY

The present disclosure is based, at least in part, unexpected discovery that the compound having formula (I):

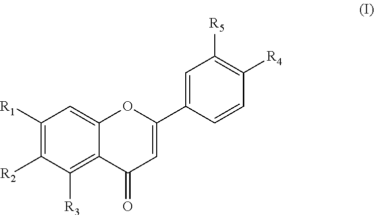

provides activity in reinstating a drug (e.g., a psycotomimetic drug such as methamphetamine or ketamine) induced disruption of prepulse inhibition (PPI) of startle reflex of a subject such as human, wherein R₁, R₂, R₃, R₄, and R₅ are independently H, OH or OCH₃. The results of this invention suggest that the efficacy of such compound may be acting through the alpha 6 subunit-containing GABA_{A} receptor within the cerebellum of the subject. Therefore, these active compounds are potential lead compounds for use as therapeutic agents for the treatment of psychiatric disorders, particularly neuropsychiatric disorders with sensorimotor gating deficits.

Accordingly, it is the first aspect of this disclosure to provide a method of treating neuropsychiatric disorders with sensorimotor gating deficits. The method comprises administering to a subject a therapeutically effective amount of a compound having formula (I):

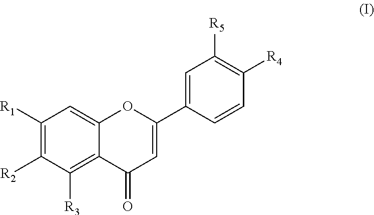

wherein R₁, R₂, R₃, R₄, and R₅ are independently H, OH or OCH₃; and the compound is effective in reinstating a drug induced disruption of prepulse inhibition (PPI) of startle reflex of the subject. In one example, R₁, R₃, and R₄ are independently OH, R₂ is OCH₃, and R₅ is H. In still another example, both R₁ and R₃ are OH; both R₂ and R₅ are H, and R₄ is OCH₃. In a further example, R₁, R₃, R₄, and R₅ are independently OH, and R₂ is H.

The subject may be a mammal, preferably a human. The neuropsychiatric disorders with sensorimotor gating deficits is any of schizophrenia, obsessive compulsive disorder (OCD), attention deficient hyperactivity disorder (ADHD) or Tourette's syndrome (TS).

In some embodiments, the dose administered to the subject is from about 1 to 100 mg/Kg body weight of the subject by injection, such as intravenous, intraperitoneal, or intra-cerebella injection. In certain embodiments, the dose is administered to the subject by intraperitoneal injection from about 10 to 100 mg/Kg body weight of the subject. In other embodiments, the dose is administered to the subject by intra-cerebella injection from about 1 to 100 nmol. The dose can be administered in a single aliquot, or alternatively in more than one aliquot.

In some embodiments, the method further comprises administering to the subject an agent that is known to improve the symptoms of neuropsychiatric disorders with sensorimotor gating deficits before, together with and/or after administering the compound having the formula shown above. Examples of such agent include, but are not limited to, tranquilizers, anti-psychotics, anti-depressants, anxiolytics, serotonin/norepinephrine/dopamine transporter inhibitors, dopaminergic agonists, anticholinergics, α2 receptor agonists, and the like.

It is therefore the second aspect of this disclosure to provide a use of the compound of formal (I) as described above for manufacturing a medicament or a pharmaceutical composition for treating neuropsychiatric disorders with sensorimotor gating deficits; the medicament or the pharmaceutical composition comprises a therapeutically effective amount of a compound having the formula shown above; and a therapeutically acceptable excipient.

The compound of this invention is present at a level of about 0.1% to 99% by weight, based on the total weight of the pharmaceutical composition. In some embodiments, the compound of this invention is present at a level of at least 1% by weight, based on the total weight of the pharmaceutical composition. In certain embodiments, the compound of this invention is present at a level of at least 5% by weight, based on the total weight of the pharmaceutical composition. In still other embodiments, the compound of this invention is present at a level of at least 10% by weight, based on the total weight of the pharmaceutical composition. In still yet other embodiments, the compound of this invention is present at a level of at least 25% by weight, based on the total weight of the pharmaceutical composition.

In some embodiments, the medicament or the pharmaceutical composition of this invention further includes an agent that is known to improve the symptoms of neuropsychiatric disorders with sensorimotor gating deficits. Examples of such agent include, but are not limited to, tranquilizers, anti-psychotics, anti-depressants, anxiolytics, serotonin/norepinephrine/dopamine transporter inhibitors, dopaminergic agonists, anticholinergics, α2 receptor agonists, and the like.

The details of one or more embodiments of the invention are set forth in the accompanying description below. Other features and advantages of the invention will be apparent from the detail descriptions, and from claims.

It is to be understood that both the foregoing general description and the following detailed description are by examples, and are intended to provide further explanation of the invention as claimed.

### BRIEF DESCRIPTION OF THE DRAWINGS

The present description will be better understood from the following detailed description read in light of the accompanying drawings, wherein:

FIG. 1 illustrates the effect of hispidulin (10 mg/Kg, i.p.) or acacetin (10 mg/Kg, i.p.) on methamphetamine (MeAmph, 2 mg/Kg, i.p.) induced hyperlocomotion activity in accordance with one embodiment of this invention;

FIG. 2 illustrates the effect of hispidulin (10 mg/Kg, i.p.) on ketamine (KET, 30 mg/Kg, i.p.) induced impairment of pre-pulse inhibition of the startle response in mice in accordance with one embodiment of this invention, wherein *p<0.05, ***p<0.001 vs control (n=8); #p<0.05 vs vehicle+KET group;

FIG. 3A and 3B illustrate the effect of hispidulin (50 mg/Kg, i.p.) or diazepam (1 mg/Kg, i.p.) on methamphetamine (MeAmph, 2 mg/Kg, i.p.) induced impairment of pre-pulse inhibition of the startle response in mice in accordance with one embodiment of this invention;

FIG. 4 illustrates the effect of bilateral intra-cerebella microinjection of hispidulin (10 nmol, i.cb.) on methamphetamin (MeAmph, 2 mg/Kg, i.p.), ketamine (KET, 30 mg/Kg, i.p.), or MK 801 (0.3 mg/Kg, i.p.) induced impairment of pre-pulse inhibition of the startle response in mice in accordance with one embodiment of this invention, wherein **p<0.01, ***p<0.001 vs control; #p<0.05, ##p<0.01 vs vehicle (i.cb.) alone in methamphetamine, ketamine, or MK-801 treated mice (n=8);

FIG. 5 illustrates the effect of bilateral intra-cerebella microinjection of diazepam (DZ) (10 nmol, i.cb), Ro-154513 (10 nmol, i.cb) and hispidulin (10 nmol, i.cb.) on ketamine (KET, 30 mg/Kg, i.p.) induced impairment of pre-pulse inhibition of the startle response in mice in accordance with one embodiment of this invention, wherein **p<0.01, ***p<0.001 vs control; #p<0.05, ##p<0.01 vs vehicle (i.cb.) alone in ketamine treated mice (n=8);

FIG. 6 illustrates the effect of bilateral intra-cerebella microinjection of furosemide (50 nmol, i.cb) on reinstatement induced by intra-cerebella microinjection of Ro-154513 (10 nmol, i.cb) and hispidulin (10 nmol, i.cb.) on methamphetamine (MeAmph, 2 mg/Kg, i.p.) and ketamine (KET, 30 mg/Kg, i.p.) induced PPI inhibition in mice in accordance with one embodiment of this invention, wherein **p<0.01, ***p<0.001 vs control (n=8); and

FIG. 7 illustrates the effect of acacetin (10 mg/Kg, i.p.) or luteolin (10 mg/Kg, i.p.) on methamphetamine (MeAmph, 2 mg/Kg, i.p.) induced impairment of pre-pulse inhibition of the startle response in mice in accordance with one embodiment of this invention; wherein *p<0.05 vs. the Vehicle-Saline group (n=8), ##p<0.01, and ###p<0.001 vs. vehicle (i.p.) alone in methamphetamine treated mice (the

Vehicle-MeAmph group) (n=6).

### DETAILED DESCRIPTION OF PREFERRED EMBODIMENTS

The detailed description provided below in connection with the appended drawings is intended as a description of the present invention and is not intended to represent the only forms in which the present invention may be constructed or utilized.

The present disclosure is based, at least in part, unexpected discovery that the compound having formula (I):

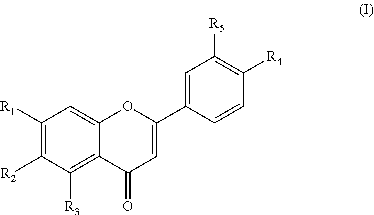

provides activity in re-instating drug induced disruption of prepulse inhibition (PPI) of startle reflex (e.g., methamphetamine or ketamine induced PPI disruption) of a subject such as human, wherein R₁, R₂, R₃, R₄, and R₅ are independently H, OH or OCH₃. The results of this invention suggest that the efficacy of such compound may be acting through the alpha 6 subunit-containing GABA_{A} receptors within the cerebellum of the subject. Therefore, these active compounds are potential lead compounds for use as therapeutic agents for the treatment of neuropsychiatric disorders with sensorimotor gating deficits.

Accordingly, this disclosure provides a method of treating neuropsychiatric disorders with sensorimotor gating deficits. The method comprises steps of administering to a subject a therapeutically effective amount of a compound having formula (I):

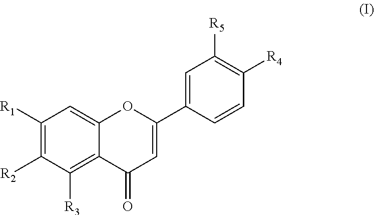

wherein R₁, R₂, R₃, R₄, and R₅ are independently H, OH or OCH₃; and the compound is effective in reinstating a drug induced disruption of prepulse inhibition (PPI) of startle reflex of the subject. In one example, R₁, R₃, and R₄ are independently OH, R₂ is OCH₃, and R₅ is H. In still another example, both R₁ and R₃ are OH; both R₂ and R₅ are H, and R₄ is OCH₃. In a further example, R₁, R₃, R₄, and R₅ are independently OH, and R₂ is H.

Shown below are exemplary compounds, compounds 1-36, of this invention.

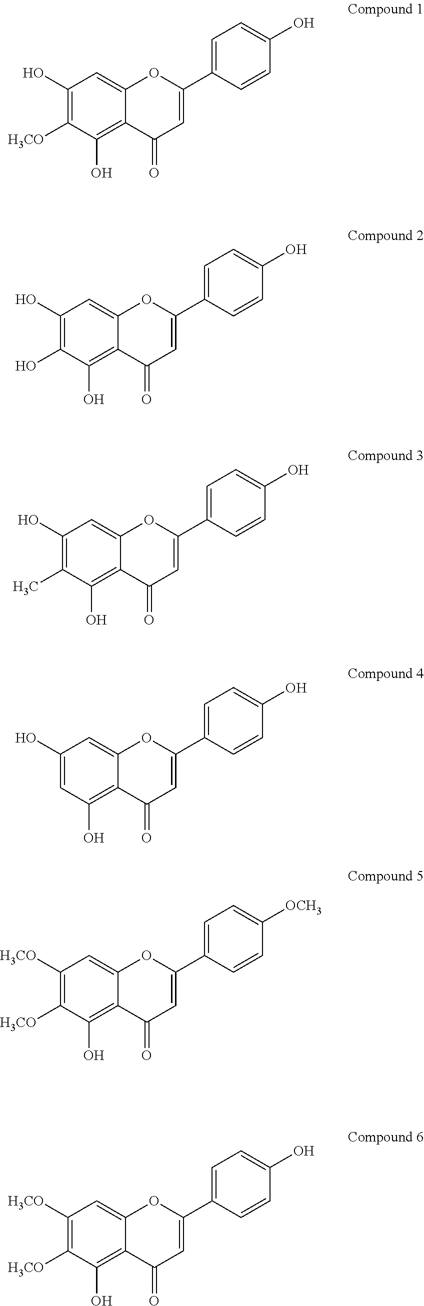

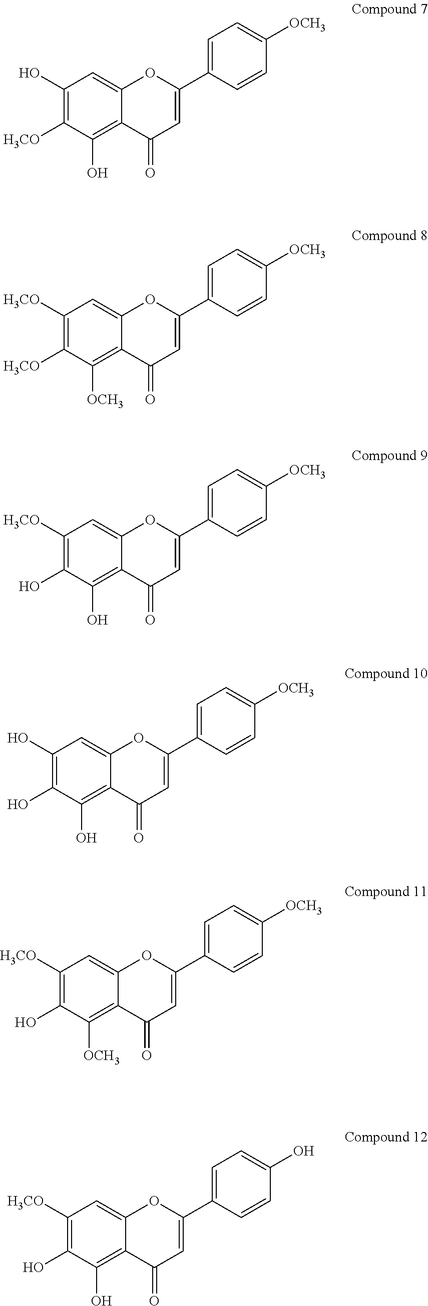

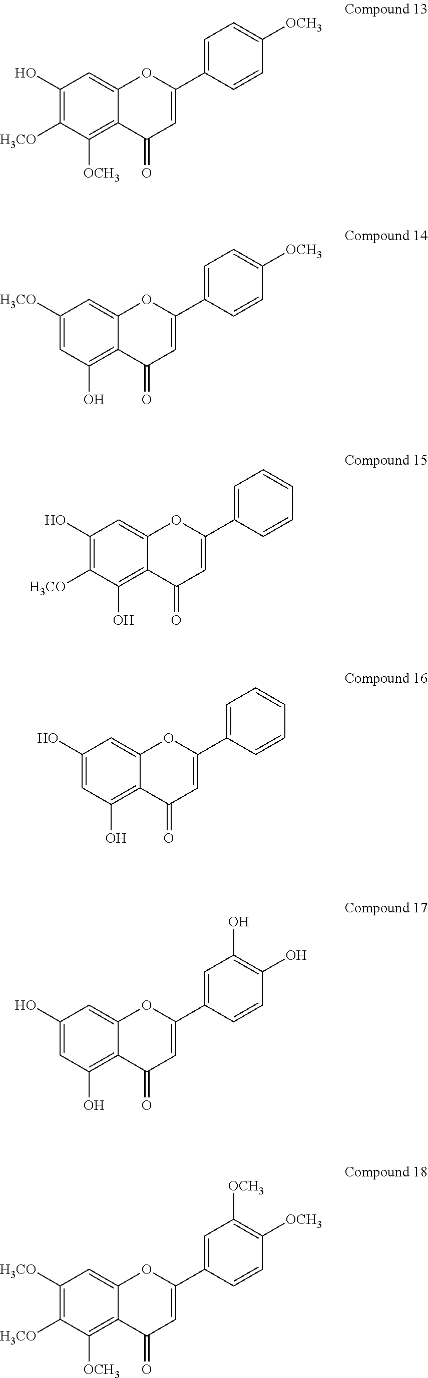

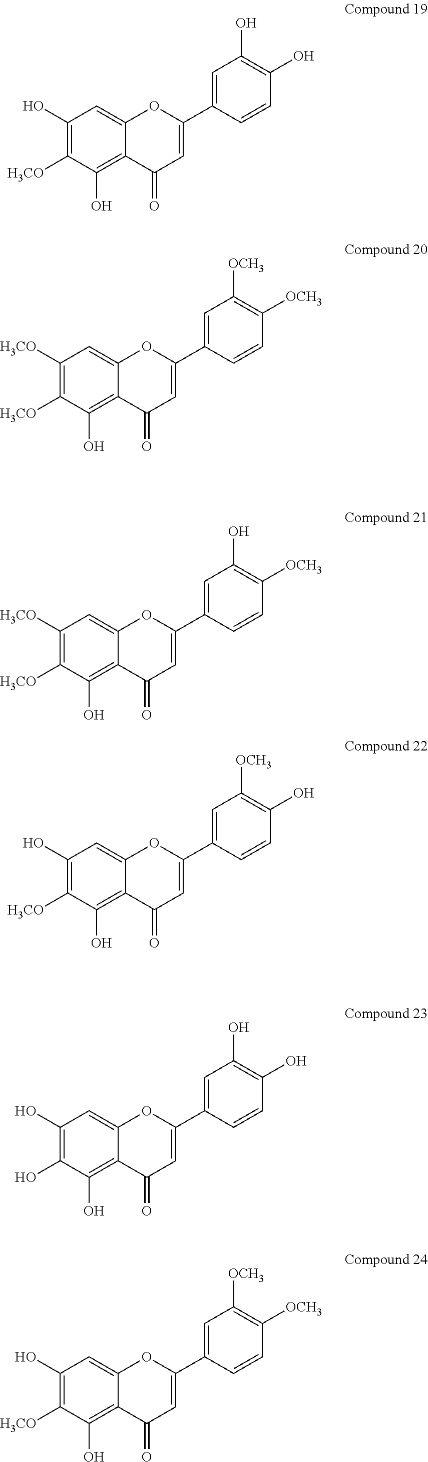

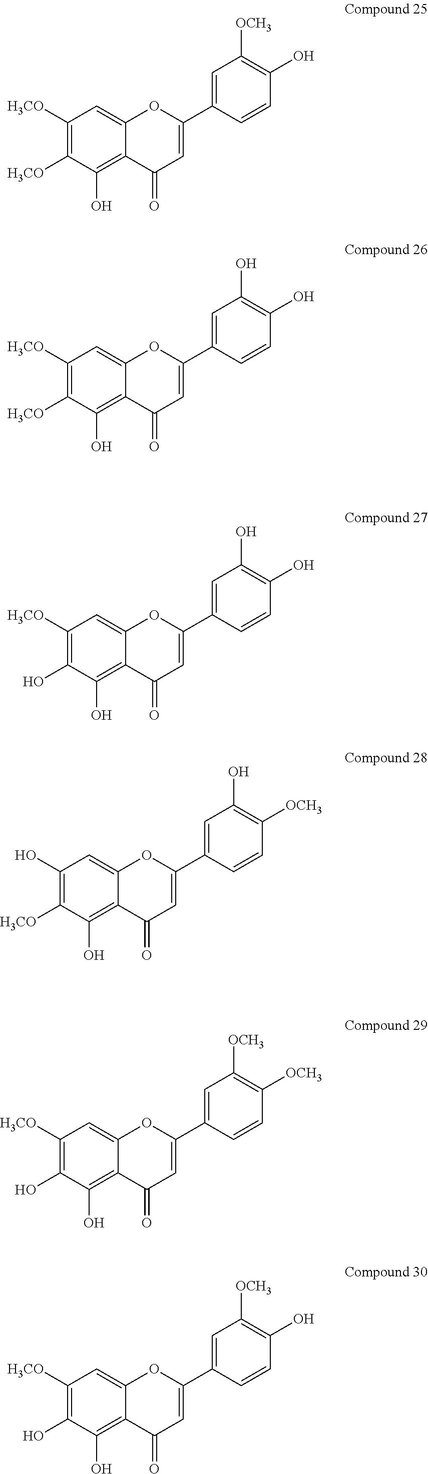

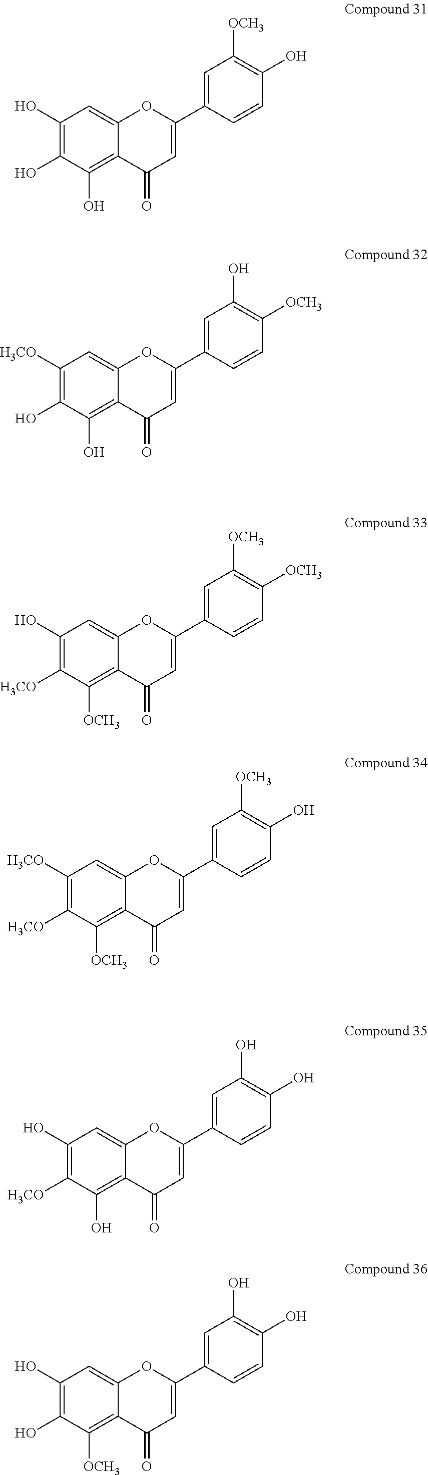

The compounds of this invention are nature flavones having 2-phenylchromen-4-one skeleton, and may be isolated from plants by purification methods known in the art, such as extraction and repetitive chromatography. For example, compound 1 (i.e., hispidulin) may be isolated from Scutellaria baicalensis or Clerodenrum inerme; compound 3 (i.e., 6-methylapigenin) may be isolated form Valeriana wallichii; compound 4 (i.e., apigenin) may be isolated from Matricaria recutita; compound 14 (i.e., acacetin) may be isolated from Robinia pseudoacacia, damiana (Turnera diffusa) or Clerodenrum inerme; compound 15 (i.e., oroxylin A) may be isolated from Scutellaria baicalensis; compound 16 (i.e., chrysin) may be isolated from Passiflora incarnate; and. As to compound 17 (i.e., luteolin), it is most commonly found in leaves, but it is also seen in celery, thyme, dandelion, rinds, barks, clover blossom and ragweed pollen. It has also been isolated from Salvia tomentosa. Dietary sources for luteolin include celery, green pepper, thyme, perilla, chamomile tea, carrots, olive oil, peppermint, rosemary, navel oranges and oregano.

Alternatively, some of the compound of formula (I) of this invention may be synthesized by ways known to those skilled in the art, for example, compound 15 (i.e., oroxylin A) may be synthesized according to the method described by Huang et al (J. Med Sci 2010 30(2), 41-46); compound 4, 16 and 17 (i.e., apigenin, chrysin and luteolin, respectively) may be synthesized according to the method described by Hutchins and Wheeler (J. Chem. Soc., 1939, 91-94). Alternatively, compound 17 or luteolin may be synthesized according to the method described in U.S. Pat. No. 6,538,021.

The subject may be a mammal, preferably a human. The neuropsychiatric disorders with sensorimotor gating deficits is any of schizophrenia, obsessive compulsive disorder (OCD), attention deficient hyperactivity disorder (ADHD) or Tourette's syndrome (TS).

In some embodiments, the dose administered to the subject is from about 1 to 100 mg/Kg body weight of the subject by injection, such intravenous or intra-cerebella injection. The dose is administered to the subject by intravenous injection at about 10, 20, 30, 40, 50, 60, 70, 80, 90 or 100 mg/Kg body weight of the subject, preferably about 50 to 70 mg/Kg body weight of the subject, such as 50, 60 or 70 mg/Kg body weight of the subject; most preferably about 50 mg/Kg body weight of the subject. The dose can be administered in a single aliquot, or alternatively in more than one aliquot.

In some embodiments, the method further includes the step of administering an agent that is known to improve the symptoms of neuropsychiatric disorders with sensorimotor gating deficits, before, together with and/or after administering the compound of this invention. Examples of such agent include, but are not limited to, tranquilizer, anti-psychotics, anti-depressants, anxiolytics, serotonin/norepinephrine/dopamine transporter inhibitors, dopaminergic receptor agonists, anticholinergics, α2 receptor agonists, and the like, that are known in the art.

This disclosure also provides a pharmaceutical composition for treating neuropsychiatric disorders with sensorimotor gating deficits; the composition comprises a therapeutically effective amount of a compound having formula (I) as shown above; and a therapeutically acceptable excipient.

Generally, the compound having formula (I) of this invention is present at a level of about 0.1% to 99% by weight, based on the total weight of the pharmaceutical composition. In some embodiments, the compound having formula (I) of this invention is present at a level of at least 1% by weight, based on the total weight of the pharmaceutical composition. In certain embodiments, the compound having formula (I) is present at a level of at least 5% by weight, based on the total weight of the pharmaceutical composition. In still other embodiments, the compound having formula (I) is present at a level of at least 10% by weight, based on the total weight of the pharmaceutical composition. In still yet other embodiments, the compound having formula (I) is present at a level of at least 25% by weight, based on the total weight of the pharmaceutical composition.

In some embodiments, the medicament of said pharmaceutical composition of this invention further includes an agent that is known to improve the symptoms of neuropsychiatric disorders with sensorimotor gating deficits. Examples of such agent include, and are not limited to, tranquilizers, anti-psychotics, anti-depressants, anxiolytics, serotonin/norepinephrine/dopamine transporter inhibitors, dopaminergic agonists, anticholinergics, α2 receptor agonists, and the like.

The medicament or said pharmaceutical composition is prepared in accordance with acceptable pharmaceutical procedures, such as described in Remington's Pharmaceutical Sciences, 17^{th} edition, ed. Alfonoso R. Gennaro, Mack Publishing Company, Easton, Pa. (1985). Pharmaceutically acceptable excipients are those that are compatible with other ingredients in the formulation and biologically acceptable.

The compounds of this invention (e.g., the compound having formula (I) as shown above) may be administered orally, parenterally, transdermally, rectally or by inhalation, alone or in combination with conventional pharmaceutically acceptable excipients. In preferred embodiments, the compounds of this invention are administered parenterally to the subject.

Applicable solid excipients may include one or more substances that may also act as flavoring agents, lubricants, solubilizers, suspending agents, fillers, glidants, compression aids, binders or tablet-disintegrating agents or an encapsulating material. In powders, the excipient is a finely divided solid that is in admixture with the finely divided active ingredient. In tablets, the active ingredient is mixed with an excipient having the necessary compression properties in suitable proportions and compacted in the shape and size desired. The powders and tablets preferably contain up to 99% of the active ingredient. Suitable solid excipient includes, for example, calcium phosphate, magnesium stearate, talc, sugars, lactose, dextrin, starch, gelatin, cellulose, methyl cellulose, sodium carboxymethyl cellulose, polyvinylpyrrolidine and the like.

The compounds of the present invention may also be formulated into liquid pharmaceutical compositions, which are sterile solutions or suspensions that can be administered by, for example, intravenous, intramuscular, subcutaneous, intraperitoneal or intra-cerebella injection. Oral administration may be either liquid or solid composition form.

The medicament or said pharmaceutical compositions of this invention may be formulated into a variety of dosage forms for topical application. A wide variety of dermatologically acceptable inert excipients well known to the art may be employed. The topical compositions may include liquids, creams, lotions, ointments, gels, sprays, aerosols, skin patches, and the like. Typical inert excipients may be, for example, water, ethyl alcohol, polyvinyl pyrrolidone, propylene glycol, mineral oil, stearyl alcohol and gel-producing substances. All of the above dosages forms and excipients are well known to the pharmaceutical art. The choice of the dosage form is not critical to the efficacy of the composition described herein.

The medicament or said pharmaceutical compositions of this invention may also be formulated in a variety of dosage forms for mucosal application, such as buccal and/or sublingual drug dosage units for drug delivery through oral mucosal membranes. A wide variety of biodegradable polymeric excipients may be used that are pharmaceutically acceptable, provide both a suitable degree of adhesion and the desired drug release profile, and are compatible with the active agents to be administered and any other components that may be present in the buccal and/or sublingual drug dosage units. Generally, the polymeric excipient comprises hydrophilic polymers that adhere to the wet surface of the oral mucosa. Examples of polymeric excipients include, but are not limited to, acrylic acid polymers and copolymers; hydrolyzed polyvinylalcohol; polyethylene oxides; polyacrylates; vinyl polymers and copolymers; polyvinylpyrrolidone; dextran; guar gum; pectins; starches; and cellulosic polymers.

The medicament or said pharmaceutical compositions of this invention may also be formulated into an inhaler aerosol formulation for drug delivery through nasal mucosal membranes. Suitable propellants and/or co-solvents for solubilizing the active agents in medicinal aerosol formulations are well known in this art. Typical propellants are hydrofluoroalkanes such as 1,1,1,2-tetrafluoroethane (HFA-134a), 1,1,1,2,3,3,3-heptafluoropropane (HFA-227ea), 1,1,1,2,2-pentafluoroethane (HFA-125), 1,1-difluoroethane (HFA-152a), difluoromethane (HFA-32) and the like. Typical co-solvents include, but not limited to, alcohols, polyols, alkoxy derivatives, fatty acid alkyl esters, polyalkylene glycols, dimethylsulphoxide and the like.

Accordingly, this invention also provides methods of treating mammals, preferably humans, for neuropsychiatric disorders with sensorimotor gating deficits, which comprises the administration of the medicament or said pharmaceutical composition of this invention that contains a compound having formula as shown above. Such medicament or composition is administered to a mammal, preferably human, by any route that may effectively transports the active ingredient(s) of the composition to the appropriate or desired site of action, such as oral, nasal, pulmonary, transdermal, such as passive or iontophoretic delivery, or parenteral, e.g., rectal, depot, subcutaneous, intravenous, intramuscular, intranasal, intra-cerebella, ophthalmic solution or an ointment. Further, the administration of the compound of this invention with other active ingredients may be concurrent or simultaneous.

In the context of this disclosure, a number of terms shall be used.

The term “treatment” as used herein includes preventative (e.g., prophylactic), curative or palliative treatment; and “treating” as used herein also includes preventative (e.g., prophylactic), curative or palliative treatment.

The term “therapeutically effective amount” as used herein refers to an amount effective, at dosages, and for periods of time necessary, to achieve the desired therapeutically desired result with respect to the treatment of neuropsychiatric disorders with sensorimotor gating deficits.

It will be appreciated that the dosage of compounds of the present invention will vary from patient to patient not only for the particular compound or composition selected, the route of administration, and the ability of the compound (alone or in combination with one or more drugs) to elicit a desired response in the patient, but also factors such as disease state or severity of the condition to be alleviated, age, sex, weight of the patient, the state of being of the patient, and the severity of the pathological condition being treated, concurrent medication or special diets then being followed by the patient, and other factors which those skilled in the art will recognize, with the appropriate dosage ultimately being at the discretion of the attendant physician. Dosage regimens may be adjusted to provide the improved therapeutic response. A therapeutically effective amount is also one in which any toxic or detrimental effects of the compound or composition are outweighed by the therapeutically beneficial effects. Preferably, the compounds or compositions of the present invention are administered at a dosage and for a time such that the number and/or severity of the symptoms are decreased.

The terms “compounds”, “compositions”, “active compounds”, “agent” or “medicament” are used interchangeably herein to refer to a compound or compounds or composition of which, when administered to a subject (human or animal) induces a desired pharmacological and/or physiological effect by local and/or systemic action.

The term “administered”, “administering” or “administration” are used interchangeably herein to refer means either directly administering a compound or a composition of the present invention, or administering a prodrug, derivative or analog which will form an equivalent amount of the active compound within the body.

The term “subject” or “patient” refers to an animal including the human species that is treatable with the compositions and/or methods of the present invention. The term “subject” or “patient” intended to refer to both the male and female gender unless one gender is specifically indicated. Accordingly, the term “subject” or “patient” comprises any mammal which may benefit from treatment of neuropsychiatric disorders with sensorimotor gating deficits.

The present invention will now be described more specifically with reference to the following embodiments, which are provided for the purpose of demonstration rather than limitation.

### EXAMPLES

### Example 1

### Both Hispidulin and Acacetin may Inhibit Methamphetamine-Induced Hyperlocomotion

Male ICR mice (25-35 g) were used in this study. They were bred, housed and maintained in the animal facility center with temperature- and humidity-controlled and 12:12 light dark cycle in National Taiwan University Medical College (NTUM) (Taiwan, R.O.C.). All animal experiments were carried out in accordance with the guidelines established by the Institutional Animal Care and Utilization Committee of the respective university.

Methamphetamine (MeAmPh, 2 mg/kg) was injected into mice to induce hyperlocomotion in the mice as a typical model for behavioral experiments.

After injection of methamphetamine, the mice were administrated by i.p. injection of vehicle only or the test compound (e.g., hispidulin (compound 1) or acacetin (compound 14)) at the dose of 10 mg/kg. After 15 minutes, the treated mice were then subjected to locomotor activity test.

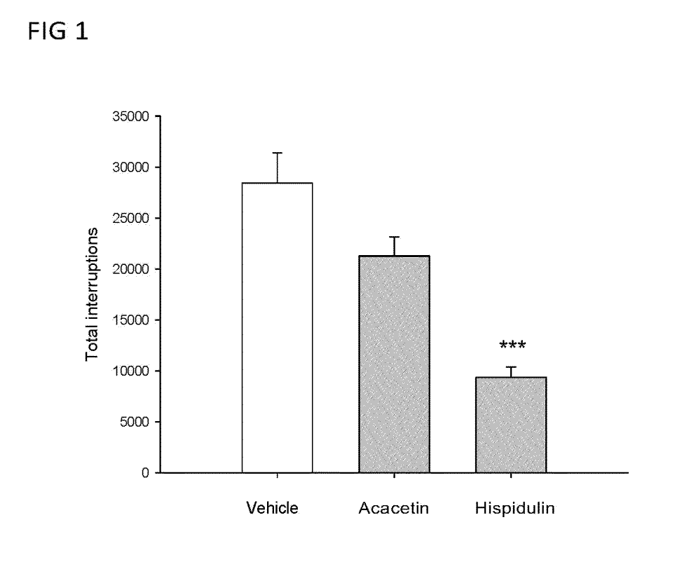

It is found that both hispidulin and acacetin were active at 10 mg/Kg (i.p.) in decreasing MeAmph-induced hyperlocomotor activity (FIG. 1).

Data herein are expressed as the mean±S.E.M and the n number indicates the number of the mice used. Statistical comparisons between the data in control and treatment conditions were analyzed by Student's t test. An effect was considered statistically significant if the p value was less than 0.05.

### Example 2

### Rescue of Methamphetamine, Ketamine or MK-801-Impaired Prepulse Inhibition (PPI) of Acoustic Startle Response by Hispidulin

Startle testing was performed in startle chambers (SR-LAB, San Diego Instruments, San Diego, Calif., U.S.A.). Each chamber consisted of a clear non-restrictive Plexglass cyclinder (5.5 cm in diameter and 13 cm in length) resting on a Plexiglass platform inside a ventilated and illuminated enclosure housed in a sound-attenuated room. A continuous background noise of 65 dB, as well as a 115 dB accoustic stimulation, was produced in the chamber by a high-frequency loudspeaker mounted 12 cm above the cylinder. The whole-body startle response of each animal produced vibratoin of the Plexiglass cylinder, which was transduced into analog signals by a piezoelectric unit, mounted underneath the Plexiglass plateform. These analog signal was then digitized and stored in a computer. Startle amplitude was defined as the degree of motion detected by the piezoelectric unit.

On the day of test, mice housed in their home cages were transferred from their holding room to a behavioral room and were acclimated for 1 hour before testing. The animals were subject to a 4-minute acclimation to the 65 dB background noise, which continued throughout the session. All PPI test sessions consisted of startle trials (PULSEALONE), prepulse trials (PREPULSE+PULSE), and no stimulus trials (NOSTIM). The test session began and ended with each four presentations of the NOSTIM trial and the PULSE-ALONE trial; in between, each fourteen acoustic or NOSTIM trial type was presented in a pseudorandom order. The inter-trial interval was randomly ranged from 5 s to 20 s. The PULSE-ALONE trials consisted of a 20 ms, 115 dB pulse of broadband noise. The PREPULSE+PULSE trials consisted of PULSE-ALONE preceded 120 ms by a 20-ms noise burst that was 71 or 77 dB. The NOSTIM trial consisted of background noise (65 dB) only. The stabilimeter recorded startle response in 100 ms beginning at stimulus onset. The startle response was defined as the value which the maximum amplitude subtracted the initial amplitude. PPI % was determined by the following equation which composing of summary startle response of each trials

(PULSEALONE−PREPULSE+PULSE)/PULSEALONE*100%

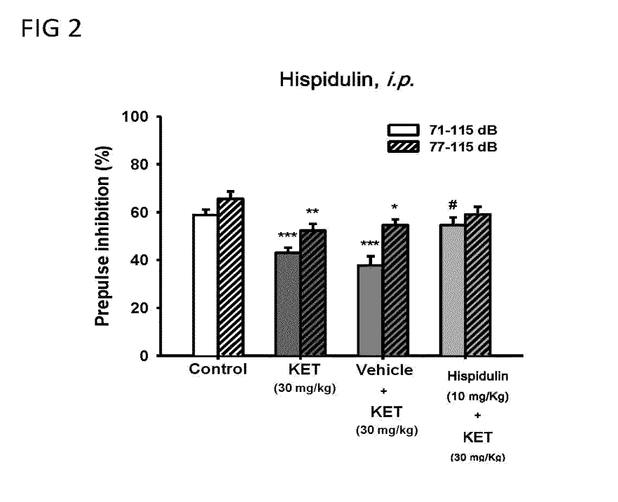

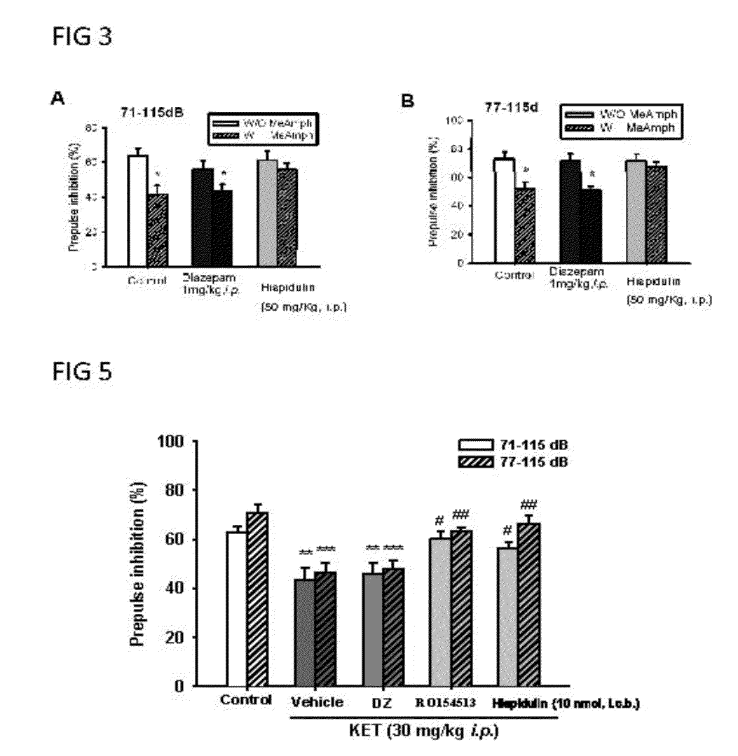

In this study, PPI was measured by the inhibition of the startle response of the mouse in response to a 115 dB acoustic stimulation when a pre-pulse acoustic stimulation at 71 dB or 77 dB was applied. In the control experiment, each animal was pre-treated with ketamine (KET, 30 mg/Kg, i.p.) or methamphetamine (MeAmph, 2 mg/Kg, i.p.) before being subjected to PPI test. As expected, both KET and MeAmph inhibited the pre-pulse inhibition (produced by 71 or 77 dB prepulse) of the startle response to a 115 dB acoustic stimulation; in other words, KET and MeAmph respectively impaired PPI inhibition. In contrast, in the case when the animal was pre-treated with hispidulin (10 mg/Kg, i.p. in FIGS. 2, and 50 mg/Kg, i.p. in FIG. 3), 15-minute before administration of KET or MeAmph, the KET or MeAmph impaired PPI inhibition was rescued (FIGS. 2 and 3).

Compound 1 or hispidulin is known to be a positive allosteric modulator of GABA_{A} receptors consisting of β², γ₂ₛ, and various α subunits, including α₁₋₃, α₅ and α₆. Further, the α₆ subunit-containing GABA_{A} receptors are exclusively expressed in the cerebellar granule cells and known to be resistant to classical benzodiazepines, such as diazepam. Hence, diazepam (1 mg/Kg, i.p.) was also administered to the test animals to see if such agent might affect MeAmph induced PPI disruption. It was found that diazepam may not rescue MeAmph impaired PPI inhibition (FIGS. 3A and 3B).

Taken together, results in FIGS. 3A and 3B suggest that hispidulin, though injected intrapertoneally, is effective in rescuring ketamine or MeAmph-impaired PPI inhibition, and the effect of hispidulin might be actingthrough, but not limited to, the α₆ subunit-containing GABA_{A} receptors in the cerebellum.

### Example 3

### Microinjection Hispidulin into Cerebella Confirms that Hispidullin Acts on Alpha 6 Subunit-Containing GABA Receptors to Rescure MeAmph-, KET or MK-801-Impaired Prepulse Inhibition of Startle Response

In view of the finding in FIGS. 2 and 3, test compounds (e.g., hispidulin or Ro-154513) were microinjected into cerebella (e.g., i.cb microinjection) of the test animals to confirm the action site of hispidulin, by observing whether the MeAmph, KET, or MK-801-induced PPI disruption may be rescued.

Briefly, the mice were anaesthetized with pentobarbital (100 mg/kg i.p.) and placed in a stereotaxic frame. After shaving the hair, the skull surface was exposed by an incision. The head position of the mice was adjusted so that the bregma-lambda axis was horizontal. Then, two 24 gauge stainless-steel cannulae were implanted bilaterally into the lateral cerebellum (−6.4 mm caudal, ±1.5 mm lateral, −1.0 mm ventral from Bregma), according to stereotaxic coordinates (Paxions, 2001). Three stainless steel screws and dental cement were used to fix the cannulae on the top of the skull. The animal was allowed to fully recover after surgery for at least one week before the behavioral experiments were conducted.

On the day of experiment, test compounds were injected through the embedded cannula at 0.5 μl on each side of the cerebellum for 30 s and waited for 1 min to allow the injected compounds to diffuse into the cerebellum. PPI test were conducted as described in Example 2. After experiments, methylene blue was microinjected (0.5 μl/side) and animals were sacrificed and cerebellar slices were dissected to confirm the microinjection site. The data was discarded in the mice with incorrect microinjection sites.

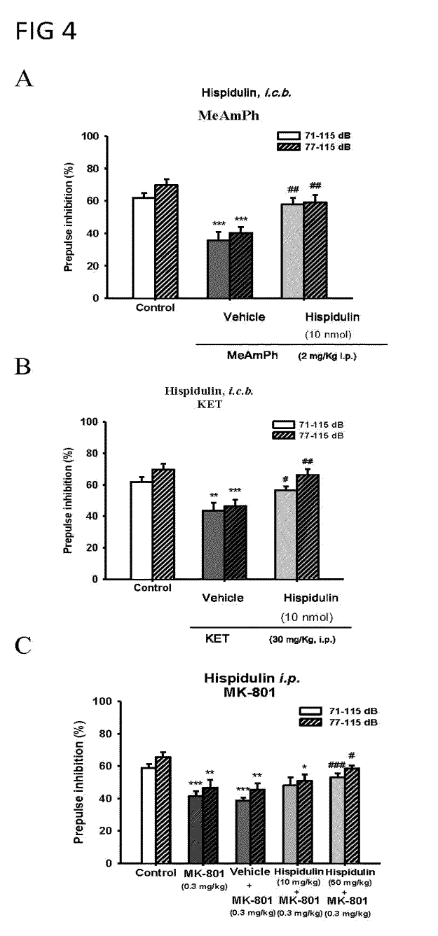

It was found that hispidulin (10 nmol, i.cb.), when microinjected into cerebella, was also effective in reinstating methamphetamine (2 mg/Kg. i.p.), ketamine (KET, 30 mg/Kg, i.p.) or MK-801 (0.3 mg/Kg. i.p.) (which is a non-competitive antagonist of N-methyl-d-asparate (NMDA) receptor) induced PPI disruption (FIG. 4). Such finding confirms that hispidulin exerts its action from within the cerebellum.

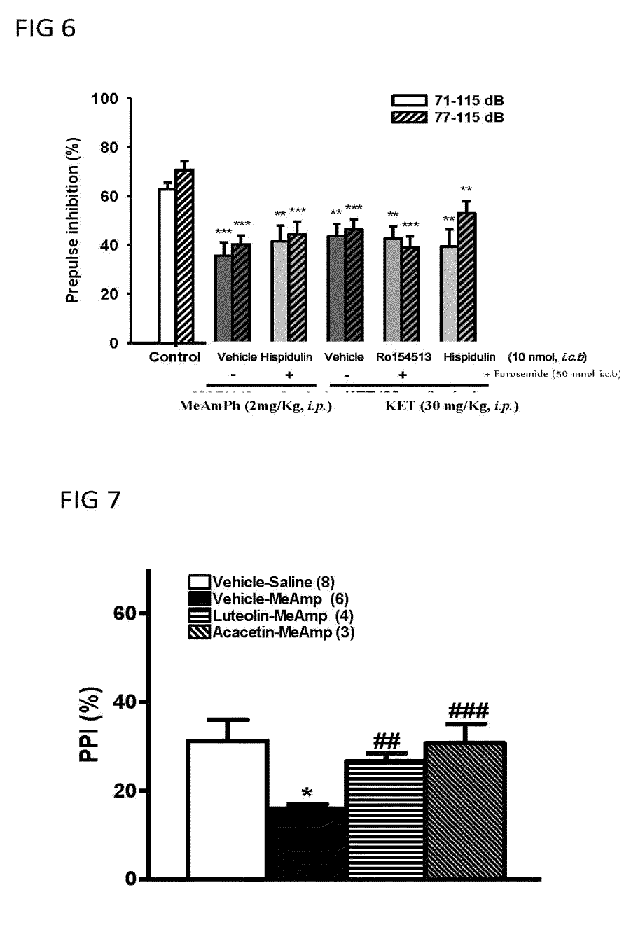

Subsequent studies found that the ketamine induced PPI disruption was reinstated by imidazobenzodiazepine Ro 154513 (10 nmol, i.cb.), an agent known to possess affinity with alpha 6 subunit-containing GABA_{A} receptors (FIG. 5). Furthermore, the reinstated ketamine (KET, 30 mg/Kg, i.p.) or methanmphetamine (MeAmPh, 2 mg/Kg. i.p.) induced PPI disruption was antagonized by bilateral intra-cerebella microinjection of furosemide (50 nmol, i.cb) (FIG. 6), which is a specific antagonist of alpha 6 subunit-containing GABA_{A} receptors. Taken together, these findings suggest that hispidulin might be acting, but not limited to, through the alpha 6 subunit-containing GABA_{A} receptors in the cerebellum.

### Example 4

### Rescue of Methamphetamine-Impaired Prepulse Inhibition (PPI) of Startle Reflex by Acacetin or Luteolin

Methamphetamin-induced PPI disruption (MeAmPh, 2 mg/kg (i.p.)) was conducted in accordance with similar steps described in Example 2 with a 71 dB-prepulse protocol. Then, the mice were administrated by i.p. injection with 10 mg/Kg (i.p.) of acacetin (i.e., compound 14) or luteolin (i.e., compound 17), and it was found that either compound may significantly rescued the PPI disruption induced by methamphetamine (FIG. 7).

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. Use of a compound having formula (I) for manufacturing a medicament for treating a psychiatric disorder with sensorimotor gating deficits, 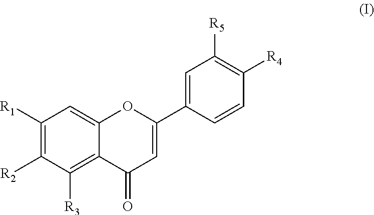
wherein R₁, R₂, R₃, R₄, and R₅ are independently H, OH or OCH₃.

2. The use of claim 1, wherein R₁, R₃, and R₄ are independently OH; R₂ is OCH₃; and R₅ is H.

3. The use of claim 1, wherein both R₁ and R₃ are OH; both R₂ and R₅ are H, and R₄ is OCH₃.

4. The use of claim 1, wherein R₁, R₃, R₄, and R₅ are independently OH; and R₂ is H.

5. The use of claim 1, wherein the psychiatric disorder with sensorimotor gating deficits is any of schizophrenia, obsessive compulsive disorder (OCD), attention deficient hyperactivity disorder (ADHD) or Tourette's syndrome (TS).

6. A method of treating a subject suffering from a psychiatric disorder with sensorimotor gating deficits comprising administering to the subject an effective amount of a compound of formula (I), 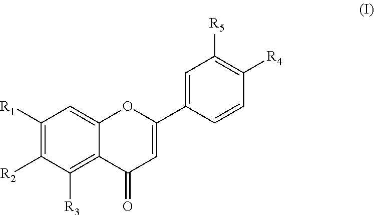
wherein R₁, R₂, R₃, R₄, and R₅ are independently H, OH or OCH₃.

7. The method of claim 6, further comprising administering to the subject an effective amount of a tranquilizer, an anti-psychotics or an anti-depressant before, together with or after administering the compound of formula (I).

8. The method of claim 6, wherein R₁, R₃, and R₄ are independently OH; R₂ is OCH₃; and R₅ is H.

9. The method of claim 6, wherein both R₁ and R₃ are OH; both R₂ and R₅ are H, and R₄ is OCH₃.

10. The method of claim 6, wherein R₁, R₃, R₄, and R₅ are independently OH; and R₂ is H.

11. The method of claim 6, wherein the psychiatric disorder with sensorimotor gating deficits is any of schizophrenia, obsessive compulsive disorder (OCD), attention deficient hyperactivity disorder (ADHD) or Tourette's syndrome (TS).

12. The method of claim 6, wherein the compound of formula (I) is administered by intraperitoneal injection.

13. The method of claim 6, wherein the subject is a human.
